(19) United States
(12) Patent Application Publication
(10) Pub. No.: US 2003/0031722 A1
(43) Pub. Date: Feb. 13, 2003

(54) Starch-oil composites for use in personal care applications

(76) Inventors: Hongjie Cao, Hillsborough, NJ (US); Gary T. Martino, Monmouth Junction, NJ (US)

Correspondence Address: Thomas F. Roland, NATIONAL STARCH AND CHEMICAL COMPANY, 10 Finderne Avenue, Bridgewater, NJ, 08807-0500, US

(21) Appl. No.: 09/873,505

(22) Filed: Jun. 4, 2001

(51) Int. Cl. A61K 9/16; A61K 9/50

(52) U.S. Cl. 424/493

(57) ABSTRACT

The present invention is directed to an aqueous personal care or cosmetic formulation containing a starch-encapsulated hydrophobic compound. These formulations are stable, and allow for formulations containing hydrophobic ingredients without the need for additional surfactants or emulsifiers. The starch-encapsulated hydrophobic compound is formed by jet cooking a mixture of the starch and the hydrophobic compound. Useful formulations include a wide variety of personal care and cosmetic formulations, including skin and hair care formulations.

### FIELD OF THE INVENTION

This invention relates to stable aqueous personal care and cosmetic formulations containing starch-encapsulated hydrophobic compounds.

### BACKGROUND OF THE INVENTION

Hydrophobic compounds, such as oils, are useful in personal care formulations, particularly cosmetic and dermatological formulations used on the skin and hair. Hydrophobic compounds are incompatible or immiscible with water, and tend to phase separate. Therefore, aqueous personal care formulations require a means of stabilization to prevent separation of the hydrophobic compounds.

Methods for stabilizing hydrophobic compounds in aqueous-based personal care formulations include solvent addition and emulsification. Solvents can be harsh on skin and hair, and have a negative environmental appeal. Emulsification involves the use of surfactants or emulsifiers. Unfortunately many surfactants and emulsifiers are known to cause skin irritation or allergic reactions.

Starch encapsulation has been used as a means of protecting sensitive ingredients. “Modified Starches: Properties and Uses”, O. B. Wurzburg, ed; 1986, CRC Press, Inc., p 141 describes the use of modified starches as encapsulating agents formed by spray-drying. The spray dried powders are water sensitive, and form emulsions in water. U.S. Pat. No. 6,045,823 discloses liposome absorbed active ingredients which are starch encapsulated by spray-drying. The encapsulated product is quickly-released in the presence of water. Typical starch-encapsulated be water-sensitive, and the encapsulation structure is unstable in water, thus they are of limited use in an aqueous composition.

U.S. Pat. Nos. 5,882,713, and 5,676,994, and WO 95/28849 issued to the United States Department of Agriculture disclose the preparation of non-separable starch-oil compositions formed by jet-cooking a mixture of starch and other materials.

Surprisingly it has been found that these stable water insoluble starch-oil compositions are suitable for use in aqueous-based personal care and cosmetic formulations. In such formulations, the starch-encapsulated hydrophobic compounds may provide additional benefits including a delivery system, a time release mechanism, a thickening mechanism, protection of the hydrophobic compound during processing and storage, retention on hair and skin, and a formulation without a greasy feel, and a formulation without a surfactant or emulsifying agent. Additionally, the starch encapsulant provides a smooth after-feel.

### SUMMARY OF THE INVENTION

The present invention is directed to an aqueous personal care or cosmetic formulation comprising a starch-encapsulated hydrophobic compound and water, wherein said starch-encapsulated hydrophobic compound is non-separable in the aqueous formulation.

The invention is also directed to a process for producing a stable aqueous personal care or cosmetic formulation containing a starch-encapsulated hydrophobic compound and water, wherein said starch-encapsulated hydrophobic compound is non-separable in the aqueous formulation.

As used herein, a non-separable starch-encapsulated hydrophobic compound in an aqueous formulation means a formulation in which the starch-encapsulated structure remains essentially intact. This is opposed to typical starch encapsulated materials which separate into the starch and the encapsulated material when in an aqueous solution. In the separable case, the starch is capable of forming an emulsion, and the system may be stable, but the encapsulation structure is destroyed in the aqueous environment. The structure of the starch-encapsulated hydrophobic compound of the present invention remains intact in an aqueous solution, and separates only with the addition of mechanical energy.

### DETAILED DESCRIPTION OF THE INVENTION

The present invention is directed to a stable aqueous personal care or cosmetic formulation containing a starch-encapsulated hydrophobic compound. The starch-encapsulated hydrophobic compound is produced by jet cooking a mixture of water, the hydrophobic compound, and starch. The encapsulated hydrophobic compound forms a stable dispersion in an aqueous medium, with the encapsulated structure remaining intact and inseparable.

Starch, as used herein, refers to both natural and chemically modified starch, and mixtures thereof. These include, but are not limited to starches derived from cereals, tubers, roots, legumes, fruits, stems or trunks. The native source can be, for example, corn, pea, potato, sweet potato, banana, barley, wheat, rice, sago, amaranth, tapioca, arrowroot, canna, and sorghum.

Chemically modified starches may also be used. Such chemical modifications are intended to include, without limitation: crosslinked, acetylated and organically esterified starches; hydroxyethylated and hydroxypropylated starches; phosphorylated and inorganically esterified starches; cationic, anionic, nonionic and zwitterionic starches; and succinate and substituted succinate derivatives of amylose-containing starch. Such modifications are known in the art, for example in Modified Starches: Properties and Uses, Ed. Wurzburg, CRC Press, Inc., Florida (1986). Starch genetic varients such as, for example, high amylose and high amylopectin starch, are also useful.

The hydrophobic compound can be one or more hydrophobic compounds or other water soluble materials known in the art for use in personal care and cosmetic applications. This includes, but is not limited to, mineral oils, oils of plant and animal origin, synthetic oils, fats, lipids, fatty acids, fatty alcohols, esters, ethers, waxes, and mixtures thereof. Typical hydrophobic compounds include, but are not limited to, jojoba oil, soybean oil, silicones, vitamins and their derivatives such as vitamin A, D, E, and K, fragrances, emollients, petrolatum, octyl methoxy cinnamate, benzophenone-3 (oxybenzone), colors, pigments, over-the-counter actives, water-insoluble polymers, antiperspirants, sun screen actives, water-insoluble solvents, and insect repellants. Other additives which can be dissolved or suspended in a hydrophobic compound may also be included in the invention as the hydrophobic compound for encapsulation, such as zinc oxide, titanium dioxide.

Encapsulation of the hydrophobic compound may be accomplished by mixing together water, starch, and the hydrophobic compound or compounds to form a homogeneous mixture, dispersion, suspension or emulsion. The mixture is then jet cooked, where the mixture is passed through a narrow orifice and the starch is instantly solubilized by contact with high pressure steam. The process is described in U.S. Pat. Nos. 5,882,713 and U.S. Pat. No. 5,676,994, which are incorporated herein by reference. The product is then dried by a means known in the art, such as by a drum dryer, spray dryer, oven, or freeze dryer. In some cases it is advantageous to use the starch encapsulated hydrophobic compound as an aqueous dispersion, directly from the jet cooker, without any drying.

When multiple hydrophobic compounds are involved in a formulation, each hydrophobic compound can be encapsulated separately, followed by a blending of the starch encapsulated hydrophobic compounds. Alternatively, the hydrophobic compounds can be blended first, followed by an encapsulation of the blend.

Steam-et cooking of the mixture under high turbulence produces drops of the hydrophobic compound(s) microencapsulated in starch thin films. These particulate materials are easily redispersed in water to form smooth, stable dispersions that are non-greasy, yet slippery to the touch. The particles are water insoluble, but water swellable.

The starch-encapsulated hydrophobic compounds contain from 1 to 80 percent by weight of the hydrophobic compound, preferably from 5 to 65 percent by weight and most preferably from 20 to 40 percent by weight, depending on the hydrophobic compound and the end-use for which it is intended.

The particle size of the starch-encapsulated hydrophobic compound can vary from 0.1 to 100 microns. Typically the particle size is from 2 to 10 microns. Different particle sizes may be used in final formulations depending on the end use. The particles may associate or agglomerate, forming clumps or clusters of many particles. It is possible to have suspended large particles visible to the eye-allowing personal care product manufacturers to adjust the aesthetic properties.

The starch-encapsulated hydrophobic compound may be formulated into an aqueous personal care or cosmetic formulation by any means known in the art. An aqueous formulation, as used herein, means a formulation containing at least 1 percent by weight of water, preferably 5 percent by weight of water, more preferably 10 percent by weight of water, even more preferably at least 15 percent by weight of water, even more preferably at least 20 percent by weight of water, and most preferably at least 40 percent by weight of water. The personal care or cosmetic formulations of the present invention include any formulation designed to contact skin or hair, including prescription and over the counter products, and those designed for both humans and animals. Examples of such products include, but are not limited to: skin and hair care products; skin and hair gels; body washes; shampoos including conditioning and anti-dandruff; hair conditioners including both leave on and rinse off; pomades; mousses; sun care products including sun screens, sun tanning products, sun tanning accelerators, sunless tanning preparations containing pigments, UVA/UVB filters, and after-sun products; insect repellants; lip gloss; massage gel; soap bars; creams; lotions; gels; deodorants and anti-perspirants; color cosmetics; facial foundations; oral care products such as toothpaste, mouthwash, whitening products and oral gels; and foot care products. The products can be in both an aerosol or non-aerosol form.

Depending on the end-use, the personal care or cosmetic formulation could contain up to 99 percent by weight of the starch-encapsulated hydrophobic compound, preferably up to 95 percent by weight, more preferably up to 90 percent by weight, even more preferably up to 85 percent by weight, even more preferably up to 80 percent by weight, more preferably up to 60 percent by weight, and most preferably up to 25 percent by weight.

Since the personal care compositions of the present invention can be formulated without surfactants, irritation and allergic reactions often associated with surfactants are eliminated. Surfactant-free formulations may reduce or eliminate skin penetration, potentially resulting in a product which would be considered milder to the skin. Personal care formulations may optionally contain emulsifiers or surfactants, but these are not required.

Personal care compositions of the present invention may also reduce irritation from hydrophobic substances known to be irritating to the skin. Such known irritants include, but are not limited to, vitamin A, vitamin E, sunscreen actives, and insect repellants. While not wishing to be bound by any theory, it is believed that the rate of skin absorption is reduced, reducing the level of irritation.

Another advantage of formulations of the present invention is that they do not feel greasy or oily, and do not leave visible residues. The hydrophobic compound, which could have an oily or greasy feel, is encapsulated and is released by the application of mechanical energy. The starch encapsulate provides a smooth after-feel, with a soft or silky feel.

Still another advantage of these formulations is that starch is a naturally-based ingredient, which can be advantageous in the personal care and cosmetic industries when a natural product is desired.

The starch-encapsulated hydrophobic compounds are stable in aqueous formulations over a wide pH range of from pH 3 to 9. The encapsulated compounds are also stable over a wide range of salt concentrations.

The combination of different core hydrophobic compounds with different starch shell materials allows for a variety of novel products. Milks, lotions and creams can be produced by dispersing the starch-oil encapsulation into different aqueous bases.

Hydrophobic compounds encapsulated with a cationically modified starch advantageously adhere to anionic substrates such as hair and skin. This the amount of contact between the hydrophobic compound and skin or hair, which aids in rinse-off and rub-off protection. This property allows for the same level of performance using less of the hydrophobic compound. Economical formulations are also possible, since the need for emulsifiers can be eliminated.

Formulations of the present invention can exhibit rub-off resistance, and a longer-lasting effect. While not being bound to any theory, it is believed that following release of the hydrophobic compound during application, the starch encapsulant may form a film at the interface between the hydrophobic compound and air or water. The protective barrier formed can serve to prevent loss of the hydrophobic compound from the skin or hair. This property is desirable in many products such as sunscreens, hair fixatives, and other products containing active ingredients. The personal care and cosmetic formulations of the present invention may also exhibit improved efficacy. Since less of the hydrophobic compound is lost, lower doses may be used to produce the desired effect.

Sunscreen formulations of the present invention can provide higher SPF values for the same amount of active ingredient, or the same SPF value for less active ingredient due to the more efficient use of the active ingredient.

The starch-encapsulated hydrophobic compound can be used in time release formulations. The hydrophobic compound is released from both dry and swelled starch encapsulant under mechanical force. Thus the hydrophobic compound can be released slowly, as needed by an action such as rubbing on the skin, combing, or the process of pressing lips together.

Particle size can be controlled over a wide range to create products having nearly invisible particles, to products containing large, visible, stably dispersed particles, which can function to increase the visual appeal of a product.

Hair conditioning compositions containing cationic waxy maize starch and jojoba oil have shown improvements in ease of wet comb compared to untreated hair. Other cationically modified starches with silicones and other emollients would display a similar functionality.

Starch-encapsulated compounds of the present invention demonstrate a thickening effect in aqueous formulations. Viscosity building has been observed when starch encapsulations are combined with commonly used surfactant systems. The starch-encapsulated hydrophobic compounds also have a thickening effect when incorporated into emulsion products.

In addition to the starch-encapsulated hydrophobic compound, the aqueous formulation of the present invention may also include other compounds typically used in personal care and cosmetic formulation. These include, but are not limited to, solvents such as acetone, methanol, ethanol, propanol, and mixtures of solvent; and water-soluble compounds such as water-soluble polymers.

The following examples are presented to further illustrate and explain the present invention and should not be taken as limiting in any regard.

### EXAMPLE 1

### Preparation of Starch-Encapsulated Hydrophobic Compounds

Various starch-encapsulated hydrophobic compounds have been prepared and evaluated for personal care applications. The excess-steam jet-cooking process as disclosed in the U.S. Pat. No. 5,882,713 and 5,676,994, and WO 95/28849 has been used for the sample preparations. Starch-encapsulated hydrophobic compounds are made in both liquid dispersion and dry form. Dried compounds are prepared by either drum drying or spray drying process.

Examples of the encapsulation samples are summarized in the table. All the examples listed contain 100 parts starch and 40 parts oil.

| | |
|---|---|
| Corn starch, soybean oil | Drum dried |
| Waxy cornstarch, soybean oil | Drum dried |
| OSA modified waxy maize, jojoba oil | 10% liquid dispersion |
| | Drum dried |
| | Spray dried |
| Waxy maize, jojoba oil | 10% liquid dispersion |
| | Drum dried |
| | Spray dried |
| QUAB 342 modified waxy maize, jojoba oil | 10% liquid dispersion |
| | Drum dried |
| | Spray dried |
| OSA modified tapioca starch, jojoba oil | 10% liquid dispersion |
| | Drum dried |
| Potato starch, jojoba oil | 10% liquid dispersion |
| | Drum dried |
| OSA modified potato starch, jojoba oil | 10% liquid dispersion |
| | Drum dried |

OSA modification is an octenyl succinic anhydride modified starch
QUAB 342 is 3-chloro-2-hydroxypropyldimethyldodecyl ammonium chloride. (Degussa)

### EXAMPLE 2

### Characteristics of Starch-lipid Encapsulations

Dispersion appearance of the starch-hydrophobe encapsulations varies with different starch base. The waxy maize based samples disperse easily and form smooth aqueous dispersions (like skin lotion/milk). The drum dried corn starch-soybean oil encapsulation sample is composed of agglomerates of encapsulation particles in the shape of flakes. These visible encapsulation particles can be suspended in clear skin or hair care products, such as skin or hair gels, body washes, or shampoos, with added visual effect.

Varying pH from 3 to 9, or adding salt to various encapsulation dispersions does not appear to impact the encapsulation stability, but it does influence how the particles associate to each other.

The viscosity of pre-dried encapsulation dispersions (5% solid) made with a few different starches is summarized in this table. As shown in this table, variation in viscosity among these samples has been observed.

| | Viscosity (cps) |
|---|---|
| Sample (all containing | (Brookfield, Spindle 3 |
| 100 parts starch and 40 parts oil) | @ 20 rpm) |
| 5% unloaded waxy maize | 42 |
| 5% waxy maize-jojoba oil encapsulation | 60 |
| 5% OSA modified waxy maize-jojoba oil | 734 |
| encapsulation | |
| 5% QUAB 342 modified waxy maize-jojoba oil | 186 |
| encapsulation | |
| 5% potato starch-jojoba oil encapsulation | 315 |
| 5% OSA modified potato starch-jojoba oil | 420 |
| encapsulation | |
| 5% OSA modified tapioca starch-jojoba oil | 658 |
| encapsulation | |

EXAMPLE 3
Emulsifier-free Moisturizing Skin Lotion with Starch-Oil Encapsulation

| | Formula A | | |
|---|---|---|---|
| | (Aqueous Base) | Formula B | Formula C |
| Ingredients | % w/w | % w/w | % w/w |
| D.I. Water | q.s. | q.s. | q.s. |
| CARBOPOL ETD 2020¹ | — | — | 0.15 |
| Triethanolamine (TEA) | — | — | q.s. to pH 5-6 |
| Glycerin | 2.0 | 2.0 | 2.0 |
| Disodium EDTA | 0.1 | 0.1 | 0.1 |
| Aloe Extract | 0.5 | 0.5 | 0.5 |
| GERMABEN II² | 1.0 | 1.0 | 1.0 |
| Starch-oil encapsulation³ | — | 5.0 | 5.0 |
| TOTAL | 100 | 100 | 100 |

¹Carbomer (B F Goodrich)
²Propylene glycol (and) diazolidinyl urea (and) methylparaben (and) propylparaben (ISP)
³OSA modified waxy corn starch-jojoba oil encapsulation (100 parts starch and 40 parts oil)

Preparation procedure: Disperse CARBOPOL in water and neutralize with TEA. Dissolve EDTA, add remaining ingredients in order and stir until uniform.

Formula A is a clear, colorless liquid (Brookfield viscosity: 8 cps; pH=4.5). Formula B is a white uniform product in the form of thin lotion/milk (Brookfield viscosity: 870 cps; pH=4.0). Formula C is a more viscous product in the form of lotion (Brookfield viscosity: 3539 cps; pH=5.0). The viscosity of the CARBOPOL ETD 2020 (0.15%) base has a very low viscosity (90 cps, pH=5.0). Therefore, a rheology synergistic effect was observed when the starch-hydrophobe encapsulation was combined with CARBOPOL. All the formulas are stable at 45° C. for 3 months.

The formulations of Example 3 demonstrate that: the starch-oil encapsulation can be simply dispersed into an aqueous formula base with stirring; a product can be produced in the form of milk/lotion/cream that contains water and oil without using emulsifiers or solvents; a thickening effect from the encapsulation. It was found that the formulations provided aesthetic enhancement—smooth skin after feel and initial formulation lubricity; no oily/greasy feel; a non-whitening film upon drying, which provides additional protection against rub-off. The encapsulated oils/actives are released when rubbing on the skin.

In this present example, the natural emollient jojoba oil is encapsulated for demonstration. One of skill in the art can see how other hydrophobic materials could be encapsulated in a similar manner, including fragrances, insect repellant, vitamins, sunscreens, silicones, or other actives.

EXAMPLE 4
Skin Massage Gel with Starch-lipid Encapsulations

| Ingredients | % w/w |
|---|---|
| D.I. Water | q.s. |
| CARBOPOL ULTREZ 10¹ | 0.5 |
| Triethanolamine | 0.16 |
| Glycerin | 2.0 |
| Menthol | 0.1 |
| Starch-lipid encapsulation² | 2.0 |
| GERMABEN II³ | 1.0 |
| | 100 |

¹Carbomer (B F Goodrich)
²Corn starch-soybean oil encapsulation (100 parts starch and 40 parts oil)
³Propylene glycol (and) diazolidinyl urea (and) methylparaben (and) propylparaben (ISP)

Preparation procedure: Disperse CARBAPOL ULTREZ 10 in water, neutralize with Triethanolamine and mix until clear gel formed. Add the remaining ingredients in order and mix until uniform. Menthol should be melted before being added to the batch.

The product is a clear colorless gel with white flakes/particles suspended in it. Upon applying on skin, the white particles in the product disappear and leave soft and smooth akin feel. Microscopic/IR study of the encapsulation particles in this product and in dry form shows that the encapsulated oil is released when mechanical force is applied.

EXAMPLE 5
Hand and Body Wash with Starch Encapsulated Emollients

| Ingredients | % w/w |
|---|---|
| D.I. Water | q.s. |
| STRUCTURE PLUS¹ | 10.0 |
| MONATERIC CAB-LC² | 10.0 |
| RHODACAL A-246L³ | 15.0 |
| MACKANATE EL⁴ | 5.0 |
| Citric acid (50%) | q.s. to pH 6.0 |
| KATHON CG⁵ | 0.1 |
| Starch-lipid encapsulation⁶ | 5.0 |
| | 100 |

¹Acrylates/aminoacrylates/C10-30 allcyl PEG-20 itaconate copolymer (National Starch and Chemical Co.)
²Cocamidopropyl Betaine (Uniqema)
³Sodium C14-16 Olefin Sulfonate (Rhodia)
⁴Disodium Laureth Sulfosuccinate (McIntyre)
⁵preservative (Robin & Haas)
⁶Any starch-oil encapsulation sample as listed in Example 2

Preparation procedure: Combine STRUCTURE PLUS with water and mix until homogeneous. Add the remaining ingredients to the batch one by one in the order listed with agitation. Mix until uniform.

When the encapsulation contains corn starch and soybean oil, the product is a clear amber colored gel with white visible flakes/particles suspended in it. When any other encapsulation sample as listed in Example 2 is used, the product is an opaque homogeneous gel. This hand and body wash with starch encapsulated emollient provides creamy foams and a smooth/conditioned skin after-feel.

EXAMPLE 6
3-in-1 Hair Gel (Styling, Conditioning, and Protecting)

| Ingredients | % w/w |
|---|---|
| D.I. Water | q.s. |
| STRUCTURE PLUS¹ | 12.5 |
| CELQUAT H-100² | 0.5 |
| DC 19³ | 0.1 |
| ARQUAD 15-25W⁴ | 1.0 |
| Glycolic acid (70%) | q.s. to pH 5.0 |
| GERMABEN II⁵ | 1.0 |
| Starch encapsulated Octyl Methoxycinnamate | 5.0 |
| | 100 |

¹Acrylates/aminoacrylates/C10-30 alkyl PEG-20 itaconate copolymer (National Starch and Chemical Co.)
²Polyquatemium-4 (National Starch and Chemical Co.)
³Dimethicone Copolyol (Dow Corning)
⁴Cetrimonium Chloride (Sutton Labs)
⁵Propylene glycol (and) diazolidinyl urea (and) methylparaben (and) propylparaben (ISP)

Preparation procedure: Using good agitation, slowly sift CELQUAT H-100 into water and mix until homogeneous. Add the STRUCTURE PLUS, DC 193, and ARQUAD 16-25W, and mix well. Add the glycolic acid and mix slowly until the solution becomes clear and viscous. Disperse starch encapsulation into the batch and mix until homogeneous.

The product provides hold (from CELQUAT H-100), conditioning benefits (from CELQUAT H-100, DC 193, ARQUAD 15-25W, and the starch encapsulation), and sun protection (from the starch encapsulated sunscreen) for the hair. The sunscreen active is simply introduced to the product without emulsification. The starch encapsulated sunscreen provides durable sun protection for the hair.

EXAMPLE 7
Cream Foundation with Starch Encapsulated Vitamins

| | Ingredients | % w/w |
|---|---|---|
| (A) | D.I. Water | q.s. |
| | POLYGLYCOL E-400¹ | 5.0 |
| | Propylene glycol | 5.0 |
| (B) | CERAPHYL 140A² | 2.2 |
| | CERASYNT Q³ | 0.9 |
| | Sorbitan stearate | 1.5 |
| | Stearyl alcohol | 0.5 |
| (C) | Iron oxide | 2.0 |
| | Titanium dioxide | 6.0 |
| | Kaolin | 7.0 |
| (D) | DC 344⁴ | 15.0 |
| (E) | GERMABEN II⁵ | 1.0 |
| (F) | Starch Encapsulated Vitamin A & E | 2.0 |
| | | 100 |

¹PEG-8 (Dow Corning)
²Decyl oleate (ISP Van Dyk)
³Glyceryl monostearate SE (ISP Van Dyk)
⁴Cyclomethicone (Dow Corning)
⁵Propylene glycol (and) diazolidinyl urea (and) methylparaben (and) propylparaben (ISP)

Preparation procedure: Combine phase A and heat to 75° C. Combine phase B and heat to 72° C.

Micronize phase C and add to phase B. Add BC to A with agitation. Cool to 50° C., add D. Then add E and F, and mix until homogeneous.

The starch encapsulated vitamins incorporate skin care benefits into this face foundation. The encapsulated vitamins may have improved stability, lasting effect, and less skin irritation.

### EXAMPLE 8

### Substantivity Test for the Cationic Modified Starch-lipid Encapsulation

Lumicrease dye method has been used to measure the substantivity of cationic modified starch-lipid encapsulation on anionic substrate, such as wool swatch, after being rinsed with water by dying them to highlight the cationic substance. The cationic starch encapsulation used in this test contains 4% QUAB 342 modified waxy maize (100 parts) and jojoba oil (40 parts). The starch encapsulation used as control contains waxy maize (100 parts) and jojoba oil (40 parts). Aqueous dispersions of 5% encapsulation samples have been used to treat cleaned wool swatches. The treated swatches are then rinsed under running water for 30 seconds. Solution of lumicrease dye is then applied to these swatches and followed by thorough rinsing. The color intensity of the treated swatches are measured by using the Hunter colorimeter. The “a” value, which represents the intensity of red color, is used to quantify and compare the color differences of treated swatches since the lumicrease dye is red in color. The higher “a” value means higher red color intensity. The average “a” value for the cationic starch encapsulation treated swatches is 17.53, and the average “a” value for the non-cationic modified starch encapsulation treated swatches is 1.99. This result indicates that the encapsulation containing cationic modified starch has very good substantivity against rinse-off on wool swatch.

### EXAMPLE 9

### Wet Combing Force Reduction for Hair Tress

Wet combing force reduction measurement is a commonly used method for objectively evaluating the conditioning attribute of hair tresses treated with conditioners. This method is used to assess the hair conditioning benefit of cationic starch encapsulation on hair tresses. This test is also used to verify the substantivity test result as explained in Example 8.

The same encapsulation samples used in Example 8 have been used for this test. A Sintech MTS Synergie 200 Tensile Tester has been used to measure the combing force of hair tresses. The hair tresses have been treated with encapsulation dispersion and then rinsed with water. Bleached hair was used in this test. The results on wet combing force reduction are summarized in the table.

| | Wet Combing |
|---|---|
| Hair treatment | Force Reduction |
| 5% dispersion of cationic waxy maize/jojoba oil | 60% |
| encapsulation | |
| 5% dispersion of waxy maize/jojoba oil | 17% |
| encapsulation | |

Significant reduction of wet combing force has been observed by treating hair with the cationic modified waxy maize/jojoba oil encapsulation. The result of this test also indicate that starch-oil encapsulation using cationic modified starch is substantive on the hair or skin even after rinse off, which means it can be used as an effective carrier and pool for the encapsulated materials. The actives incorporated into hair or skin care products, especially in rinse-off products, will stay and function.

EXAMPLE 10
Shampoo with Cationic Starch-Jojoba Oil Encapsulation

| | Ingredients | % w/w |
|---|---|---|
| (A) | Starch-lipid Encapsulation¹ | 5.00 |
| | D.I. Water | q.s. |
| (B) | STANDAPOL EA-2² | 35.00 |
| | REWOTERIC AM B14³ | 7.00 |
| | MONAMID 716⁴ | 1.00 |
| | MACKSTAT DM⁵ | 1.00 |
| | | 100 |

¹Cationic Waxy Maize Starch/Jojoba Oil Encapsulation
²Ammonium Laureth Sulfate (Henkel Corporation)
³Cocamidopropyl Betaine (Witco Chemical Co.)
⁴Lauramide DEA (Mona Industries)
⁵DMDM Hydantom (Mcyntyre)

Preparation procedure: Phase A: Disperse the starch/lipid in water with agitation. Phase B: In a separate vessel, using slow agitation, mix the remaining ingredients in the order listed.

Add phase A to phase B and mix until uniform.

Evaluation of this shampoo vs. the shampoo base without the encapsulation shows that this cationic starch encapsulation contributes to tighter, more creamy foams, and smooth and conditioned after feel. The addition of this encapsulation sample increased the viscosity of the shampoo base from 10 cps to 7600 cps (viscosity taken four days after formulation using Brookfield viscometer with spindle 4 @20 rpm).

EXAMPLE 11
Hair Conditioner with Cationic Starch-Jojoba Oil Encapsulation

| Ingredients | % w/w |
|---|---|
| D.I. Water | q.s. |
| CELLOSIZE Polymer PCG-10¹ | 0.6 |
| Hydrolyzed protein | 0.3 |
| ARQUAD 15-25W² | 1.0 |
| DL-PANTHENOL USP³ | 0.5 |
| GERMABEN II⁴ | 1.0 |
| Starch-lipid Encapsulation⁵ | 5.00 |
| | 100 |

¹Hydroxyethylcellulose (Amerchol)
²Cetrimonium Chloride (Sutton Labs)
³Panthenol (Roche)
⁴Propylene glycol (and) diazolidinyl urea (and) methylparaben (and) propylparaben (ISP)
⁵Cationic Waxy Maize Starch/Jojoba Oil Encapsulation

Preparation procedure: Disperse CELLOSIZE into water with propeller agitation and mix until fully hydrated. Add remaining ingredients one by one in the order listed with agitation.

The product is an easy to make rinse-off hair conditioner. The substantive cationic waxy maize starch encapsulated jojoba oil is incorporated into this product. Both the cationic waxy maize starch and the jojoba oil provide conditioning benefits to the hair.

Since the Starch-lipid encapsulation can be added into conditioner bases easily, a two-components hair conditioning system may be used by allowing the consumers/salons to choose and add the required type/amount of starch encapsulated conditioner to a conditioner base and thus to achieve adjustable conditioning effect.

Example 12
Emulsifier-free Sunscreen Cream with Starch-Sunscreen Encapsulation

| | Formula A | Formula B | Formula C | Formula D |
|---|---|---|---|---|
| Ingredients | % w/w | % w/w | % w/w | % w/w |
| D.I. Water | q.s. | q.s. | q.s. | q.s. |
| CARBOPOL | — | — | 0.15 | 0.15 |
| ETD 2020 | | | | |
| Triethanol- | — | — | q.s. to pH 5-6 | q.s. to pH 5-6 |
| amine | | | | |
| 1,3-Butylene | 2.0 | 2.0 | 2.0 | 2.0 |
| Glycol | | | | |
| Disodium | 0.1 | 0.1 | 0.1 | 0.1 |
| EDTA | | | | |
| Aloe Extract | 0.5 | 0.5 | 0.5 | 0.5 |
| GERMABEN | 1.0 | 1.0 | 1.0 | 1.0 |
| II | | | | |
| Starch-oil | 20.0 | — | 20.0 | — |
| encapsulation¹ | | | | |
| Starch-oil | — | 8.0 | — | 8.0 |
| encapsulation² | | | | |
| Starch-oil | — | 5.0 | — | 5.0 |
| encapsulation³ | | | | |
| Starch-oil | — | 6.0 | — | 6.0 |
| encapsulation⁴ | | | | |
| Starch-oil | — | 6.0 | — | 6.0 |
| encapsulation⁵ | 100 | 100 | 100 | 100 |

¹Starch encapsulated sunscreen active blend containing Benzophenone-3 (oxybenzone), Octyl methoxycinnamate, Octyl Salicylate, and Avobenzone (Parsol 1789)
²Starch encapsulated Octyl methoxycinnamate
³Starch encapsulated Octyl Salicylate
⁴Starch encapsulated Benzophenone-3 and Finsolv TN (C₁₂₋₁₅ alkyl benzoate, Fintex)
⁵Starch encapsulated Avobenzone (Parsol 1789) and Finsolv TN
Preparation procedure: Formulas A and B: Dissolve EDTA, add remaining ingredients in order and stir until uniform. Formulas C and D: Disperse CARBOPOL in water and neutralize with TEA. Dissolve EDTA, add remaining ingredients in order and stir until uniform.

What is claimed is:

1. A stable, aqueous personal care or cosmetic formulation comprising:
a) a starch-encapsulated hydrophobic compound; and
b) water,
wherein said starch-encapsulated hydrophobic compound is essentially non-separable in said aqueous formulation.

2. The aqueous formulation of claim 1 wherein said starch-encapsulated hydrophobic compound is formed by jet cooking a mixture of the starch and the hydrophobic compound.

3. The aqueous formulation of claim 1 wherein said starch is a modified starch.

4. The aqueous formulation of claim 1 wherein said starch is a natural starch.

5. The aqueous formulation of claim 3 wherein said starch is a hydrophobically modified starch.

6. The aqueous formulation of claim 3 wherein said starch is cationically modified.

7. The aqueous formulation of claim 3 wherein said starch is selected from the group consisting of crosslinked, acetylated and organically esterified starches; hydroxyethylated and hydroxypropylated starches; phosphorylated and inorganically esterified starches; cationic, anionic, nonionic and zwitterionic starches; and succinate and substituted succinate derivatives or amylose-containing starch.

8. The aqueous formulation of claim 1 wherein said hydrophobic compound is selected from the group consisting of mineral oils, oils of plant and animal origin, synthetic oils, fats, lipids, fatty acids, fatty alcohols, esters, ethers, wax, jojoba oil, soybean oil, silicones, vitamins such as vitamin A, D, E, and K, fragrances, emollients, petrolatum, colors, pigments, over-the-counter actives, water-insoluble polymers, antiperspirants, sun screen actives, benzophenone-3 (oxybenzone), octyl methoxy cinnamate, water insoluble solvents, insect repellants, and mixtures thereof.

9. The aqueous formulation of claim 1 comprising at least 0.1 percent by weight and up to 99 percent by weight of said starch-encapsulated hydrophobic compound

10. The aqueous formulation of claim 9 comprising 0.5 to 25 percent by weight of said starch-encapsulated hydrophobic compound.

11. The aqueous formulation of claim 1 comprising at least 1 percent by weight of water.

12. The aqueous formulation of claim 1 comprising at least 5 percent by weight of water.

13. The aqueous formulation of claim 1 comprising at least 10 percent by weight of water.

14. The aqueous formulation of claim 10 comprising at least 15 percent by weight of water.

15. The aqueous formulation of claim 11 comprising at least 20 percent by weight of water.

16. The aqueous formulation of claim 11 comprising at least 40 percent by weight of water.

17. The aqueous formulation of claim 1 wherein said starch-encapsulated hydrophobic compound comprises from 1 to 80 percent by weight of at least one hydrophobic compound.

18. The aqueous formulation of claim 14 wherein said starch-encapsulated hydrophobic compound comprises from 5 to 65 percent by weight of a hydrophobic compound.

19. The aqueous formulation of claim 15 wherein said starch-encapsulated hydrophobic compound comprises from 20 to 40 percent by weight of at least one hydrophobic compound.

20. The aqueous formulation of claim 1 wherein formulation is surfactant-free.

21. The aqueous formulation of claim 1 wherein said formulation is a sun care product.

22. The aqueous formulation of claim 1 further comprising a solvent.

23. The aqueous formulation of claim 1 further comprising a water-soluble polymer.

24. A process for formulating a personal care composition comprising:
a) combining water, a starch, and a hydrophobic compound to form a suspension or emulsion;
b) steam-jet cooking the combination to form a starch-encapsulated hydrophobic compound; and
c) formulating said starch-encapsulated hydrophobic compound into an aqueous cosmetic or personal care formulation.

22. The process of claim 21 further comprising drying said starch-encapsulated hydrophobic compound after jet-cooking and prior to formulation.
